(19) United States
(12) Patent Application Publication
(10) Pub. No.: US 2024/0335574 A1
(43) Pub. Date: Oct. 10, 2024

(54) Lipid Emulsion Alginate Microspheres

(71) Applicant: Board of Regents, The University of Texas System, Austin, TX (US)

(72) Inventors: Ryan Bitar, New Haven, CT (US); William Phillips, San Antonio, TX (US)

(21) Appl. No.: 18/294,090

(22) PCT Filed: Aug. 2, 2022
(86) PCT No.: PCT/US22/39221

§ 371 Date: Jan. 31, 2024

Related U.S. Application Data
(60) Provisional application No. 63/228,215, filed on Aug. 2, 2021.

(51) Int. Cl. A61K 51/12; A61K 41/00; A61K 49/00; A61K 49/04

(52) U.S. Cl. CPC A61K 51/1265; A61K 41/00; A61K 49/0091; A61K 49/0438

(57) ABSTRACT

Certain embodiments are directed to Lipid Emulsions in Alginate Microspheres, e.g., Lipiodol Emulsion in Alginate Microspheres (LEAMs), as a chemoembolic agent. A oil in water emulsion of lipiodol can be stabilized within alginate microspheres at very high concentrations. These emulsion in alginate microspheres can be manufactured to any size range from 20 microns to 5000 microns. In certain aspects, an ultrasonic nozzle technology is used to manufacture small size microspheres of average diameter 50 microns. In other aspects, needle extrusion systems are used to manufacture large LEAMs termed Mega-LEAMs.

### RELATED APPLICATION

This application is an International Application claiming priority to U.S. Provisional Patent application Ser. No. 63/228,215 filed Aug. 2, 2021, which is incorporated herein by reference in its entirety.

### STATEMENT REGARDING FEDERALLY FUNDED RESEARCH

None.

### BACKGROUND

Microspheres composed of synthetic materials have been in clinical use for interventional radiology applications to embolize tumors (Sheth et al. J Funct Biomater. 2017, 8; Hu et al. Adv Mater. 2019, 31: e1901071), deliver drugs to liver tumors (Fuchs et al. J Control Release. 2017, 262:127-38), and to stop bleeding at gastrointestinal and other sites in the human body (Shi et al. Medicine (Baltimore). 2017, 96: e9437). A recent interest has been to make microspheres that are inherently radio-opaque so that they can be visualized during intra-arterial administration and after treatment via CT imaging to precisely confirm their distribution in the liver (Vollherbst et al. PLoS One. 2018, 13: e0198911). A commercial formulation of radio-opaque microspheres composed of synthetic materials known as LC Bead LUMI™ was approved for clinical use by the FDA Dec. 14, 2015 (Reicher et al. Cardiovasc Intervent Radiol. 2019, 42:1563-70). These beads are composed of a synthetic polymer, polyvinyl alcohol acrylamido-2 methylpropane sulfonic acid hydrogel. Iodinated residues were covalently bound to the surface of the microsphere during their synthesis to confer radiopacity.

Lipiodol presents the advantages of being radiopaque and as a carrier for hydrophobic drugs and may be emulsified in combination with aqueous therapeutic agents; however, free (nonencapsulated) lipiodol presents the possibility of complications such as migratory fat emboli and/or thyrotoxicity from dissociated iodine. For example, lipiodol when injected intra-arterial, is only partially retained at the distal end of the arteriole, and a significant amount of lipiodol escapes and moves to the subsequently encountered capillary bed such as the lungs. When lipiodol was radiolabeled with I-131 and injected intra-arterially into sarcoma tumors of the extremities, the main complication was that lipiodol escaped from the tumor and went to the lungs (Richardson et al., Can Med Assoc J. 1966, 94:1086-91). To limit lung activity, patients had to remain at bedrest without extremity movement for several days.

There remains a need for additional compositions and methods for visualizing particles during intra-arterial administration and after administration to a subject. Additionally, there remains a need to delivery high quantities of lipophilic drugs in a microspheres.

### SUMMARY

Certain embodiments are directed to Radio Opaque Emulsion in Alginate Microspheres, e.g., Lipiodol Emulsion in Alginate Microspheres (LEAMs), as a chemoembolic agent. A water in oil emulsion of lipiodol or other radio-opaque composition can be stabilized within alginate microspheres at very high concentrations. The term “emulsion” as used herein refers to a dispersion of oil in water (“o/w”). The term “oil” refers to any hydrophobic substance which is insoluble or very sparingly soluble in water and which can be made into a stable aqueous emulsion of the oil-in-water type, optionally using a surfactant. The emulsion is a biphasic composition comprising oil droplets dispersed in a continuous aqueous phase. In certain embodiments, the term “emulsion” refers to continuous aqueous phase that contains a discontinuous organic phase wherein the discontinuous phase comprises discrete micelles. These emulsions in alginate microspheres can be manufactured to any size range from 20 microns to 5000 microns. In certain aspects, an ultrasonic nozzle technology (e.g., SONO-TEK™ ultrasonic nozzle) is used to manufacture small size alginate microspheres of average diameter 20-60 microns. In other aspects, needle extrusion systems (e.g., from 16 to 21 gauge needle extrusion system) are used to manufacture large LEAMs termed Mega-LEAMs which can be used for radiographic placement and localization of sites during surgery.

Lipiodol is a hydrophobic substance consisting of ethiodized fatty acids derived from poppy seed oil. The iodinated fatty acids are thus radiopaque. While the onsite synthesis of lipiodol is most likely feasible, it is a commercially available agent. Lipiodol has been used for liver tumor embolization (Lencioni et al. Hepatology. 2016, 64:106-16), and is frequently radiolabeled with I-131 to deliver radionuclide therapy (Furtado et al., Ann Surg Oncol. 2014, 21:2700-07).

Alginate is a polysaccharide derived from seaweed consisting of 2 monosaccharide residues: Mannuronate and Guluronate. Sodium alginate salt (a commercially available product) when made into an aqueous solution becomes a viscous hydrogel. When the solution comes into contact with divalent cations (such as Ca2+) cross-linkage occurs forming a hardened gel matrix (Bruchet and Melman, Carbohydr Polym. 2015, 131:57-64).

Certain embodiments are directed to an emulsion containing alginate microspheres comprising a crosslinked alginate microsphere containing an oil in water emulsion portions encapsulated by the crosslinked alginate. In certain aspects the emulsion includes a surfactant. The surfactant can be, but is not limited to polyoxyethylene sorbitan monooleate (TWEEN 80®), oleyl polyoxyiglycerides, polyoxyethylated castor oil, caprylic capric triglycerides, glyceryl monocaprylocaprate, and mixtures thereof. In certain aspects, the surfactant is present at an amount of 0.001, 0.005, 0.01, 0.05, 0.1, 0.5, to 1 weight percent, including all values and ranges there between. In certain aspects the oil is a fatty acid. In particular aspects the fatty acid is an ethiodized fatty acid. The ethiodized fatty acid can be lipiodol or a similar ethiodized fatty acid. The emulsion can be 20, 25, 30, 35, 40, 45, 50, 55, 60, 65, 70, 75, to 80 wt % oil, including all values and ranges there between. The iodine moiety can include iodine 127, iodine 131, iodine 125, or combinations thereof.

The alginate microspheres can have an average diameter of 20 microns to 5000 microns, including all values and ranges there between. In certain aspects, the microspheres have an average diameter of 2 to 5 millimeters. In other aspects, the microspheres have an average diameter of 60+/−10 microns.

The alginate microsphere can further include a hydrophobic solute in the hydrophobic phase of the emulsion portion. The hydrophobic solute can be a drug or dye. The hydrophobic dye can be a fluorescent dye. In certain aspects, the drug is a radionuclide or chemotherapeutic. The radionuclide can be a hydrophobic radionuclide chelate.

The alginate microsphere can further comprise a hydrophilic solute in the emulsion portion. In certain aspects, the hydrophilic solute is a drug or dye. The hydrophilic dye can be a fluorescent dye. The drug can be a radionuclide or chemotherapeutic. In certain aspects, the radionuclide is a hydrophilic radionuclide chelate. In certain aspects the drug is a hydrophilic or hydrophobic therapeutic drug.

Certain embodiments are directed to an alginate microsphere comprising a crosslinked alginate microsphere containing a radiopaque emulsion encapsulated by the crosslinked alginate. The emulsion can include a fatty acid. The fatty acid can be an ethiodized fatty acid. In certain aspects, the ethiodized fatty acid is lipiodol or a substitute thereof. The iodine moiety comprises iodine 131 or other iodine isotope. The iodine moiety can be a mixture of iodine isotopes.

Certain embodiments are directed to methods for producing an alginate microsphere encapsulating a radiopaque emulsion comprising: (i) preparing an ethiodized fatty acid emulsion; (ii) homogenizing the emulsion in an alginate solution; and (iii) forming the alginate microsphere encapsulating a radiopaque emulsion microsphere by contacting emulsion droplets with a calcium chloride or other alginate crosslinking solution. In certain aspects, the ethiodized fatty acid is lipiodol. In certain aspects, the step of forming the alginate microsphere includes ultrasonic atomization of the emulsion in alginate solution.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 depicts LEAMs under fluoroscopy. Falcon tubes on the left contain micron-sized (40-80 micron) LEAMs which may be employed for chemoembolization of liver tumors. The vial in the middle contains Mega-LEAMs, which are intended to be injected percutaneously into tumors for later localization during surgical resection. Fourth vial contains water, and fifth vial contains pure lipiodol.

FIG. 2A-2C depicts an example of a manufacturing method. (A) Left syringe contains 1 ml doxorubicin+1 ml of alginate solution (aqueous phase), right syringe contains 1 ml of lipiodol stained with Sudan Red (oil phase). (B) Two phases are emulsified via rapid syringe exchange. (C) Left vial contains water, right vial contains emulsion spheres produced by manually dripping into CaCl₂) solution through a 26 gauge needle. Lack of turbidity of the supernatant indicates excellent encapsulation.

FIG. 3A-3B. FIG. 3A shows the size distribution of alginate microspheres containing a fluorescent lipophilic dye manufactured with the alginate. FIG. 3B shows a light microsphere image of the microspheres.

FIG. 4A-4C. FIG. 4A shows an image of fluorescently labeled 50 micron microspheres injected intra-arterially into an ex-vivo kidney. The kidneys vessels are well demarcated. FIG. 4B shows a sagittal computed tomography (CT) images of radiocontrast lipiodol containing 50 micron microspheres injected intra-arterially into two different kidney segments. The radiographic CT images clearly show the high-density microspheres that have embolized two different segments. FIG. 4C shows a volume rendered CT images showing the arterial vascular system of the kidney administered the lipiodol radiographic contrast within the alginate microspheres.

FIG. 5 shows the set-up for manufacturing the lipophilic containing microspheres with the ultrasonic nozzle.

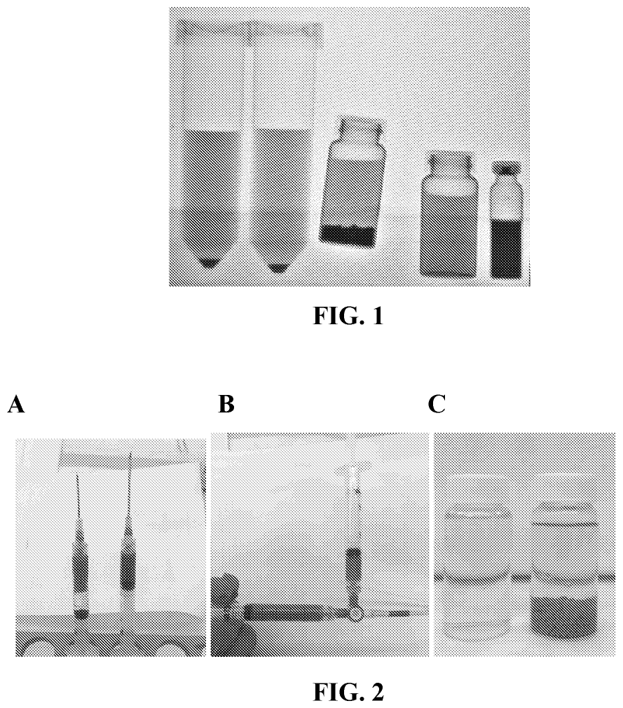

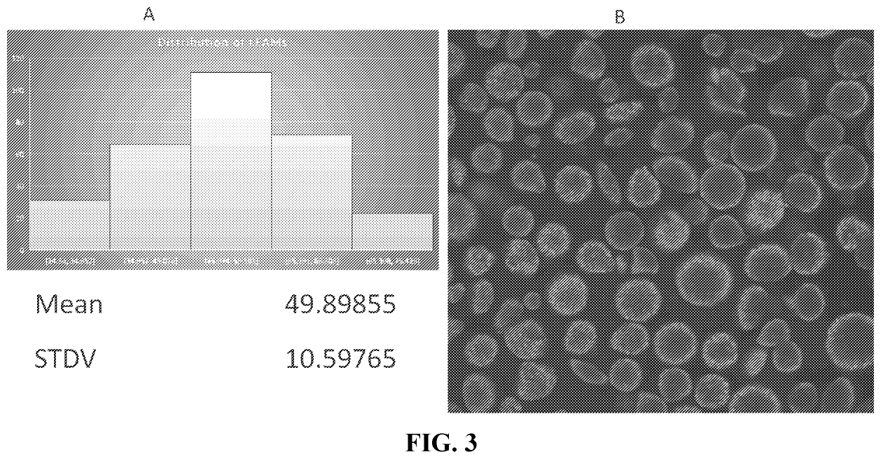

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Alginate microspheres can be manufactured to contain emulsions of radiographic contrast agents such as lipiodol (see FIG. 2 for an example). Lipiodol is a clinically-used, commercially radiographic contrast agent that is generally safe for use in humans. Because of their radiopacity provided by their lipiodol component, lipiodol emulsion in alginate microspheres (LEAMs) can be used for radiographic visualization of their distribution within a human or animal body. This visualization is possible due to the high density of the iodinated molecules which provide excellent radiographic contrast. Emulsion alginate microspheres can contain a high concentration of lipiodol or other radiopaque agents and retain it without it seeping from the porous interface of the alginate. Alginate microspheres can stably contain a very high concentration of lipiodol within microspheres. Lipiodol and other lipophilic solutions cannot simply be mixed with alginate solution and will not be retained within alginate microspheres. The process involves the surprising result that oil of the lipiodol or a lipophilic drug must be emulsified with an aqueous phase otherwise it will not remain stable within the microsphere. These results also show a surprising result that use of an ultrasonic nozzle does not disrupt the lipophilic emulsion and it remains contained within the alginate microsphere.

In certain aspects an emulsion is prepared and then homogenized in an alginate solution. Microspheres are formed by ultrasonically atomized emulsion droplets making contact with a calcium chloride solution (or other crosslinking agent) which causes crosslinking in a gentle process that is not damaging to drugs or biologic agent. Following cross-linking with the Ca2+ ions, the droplets are stabilized. The inventors have also determined that it is possible to dissolve hydrophobic solutes such as drug or dyes into the emulsion phase. Radionuclides can be chelated to hydrophobic chelators which can partition to the oil phase. Hydrophilic nanoparticles or drugs can be encapsulated into the aqueous emulsion phase of the alginate microsphere. The concept of oil-in-water emulsion alginate microspheres is not limited to LEAMs (lipiodol as the oil phase); other oils may be incorporated into spheres and may serve as stable carriers of hydrophobic compounds.

Emulsions. Certain embodiments include from about 10, 20, 30, 35, 40, 45, 50 to 60% of an emulsifiable C4-C22 vegetable oil selected from the group consisting of soybean oil, corn oil, coconut oil, rapeseed (canola) oil, peanut oil, sunflower oil, olive oil, crambe oil, and combinations thereof; about 60-35% of a non-ionic surfactant/co-surfactant blend to help to reduce the oil droplet size when compare to the use of a single surfactant and 0-12% water. Suitable surfactants include Tween 80® (polyoxyethylene sorbitan monooleate, Labrafil M1994CS® (oleyl polyoxyiglycerides), Cremophor EL® which is now called Colliphor EL® (polyoxyethylated castor oil), WAGLINOL 9238® (caprylic capric triglycerides) and CAPMUL® MCM series (glyceryl monocaprylocaprate) and mixtures thereof; and about 0-12% water. A suitable surfactant/co-surfactant blend includes Tween 80 (polyoxethylene sorbitan monooleate)/Labrafil (oleyl polyoxylglycerides) in the ratio of 2:1 mixed with Cremophor EL (polyoxyl 35 castor oil)/Waglinol (caprylic capric triglycerides) in the ratio 1:2. This blend will produce a nanoemulsion with droplets sizes below 200 nm upon gentle agitation with water. Glycerol may also be used as a co-surfactant.

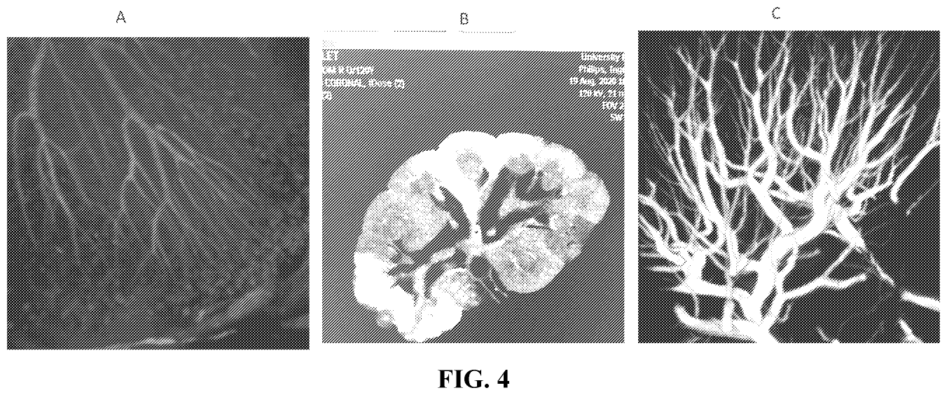

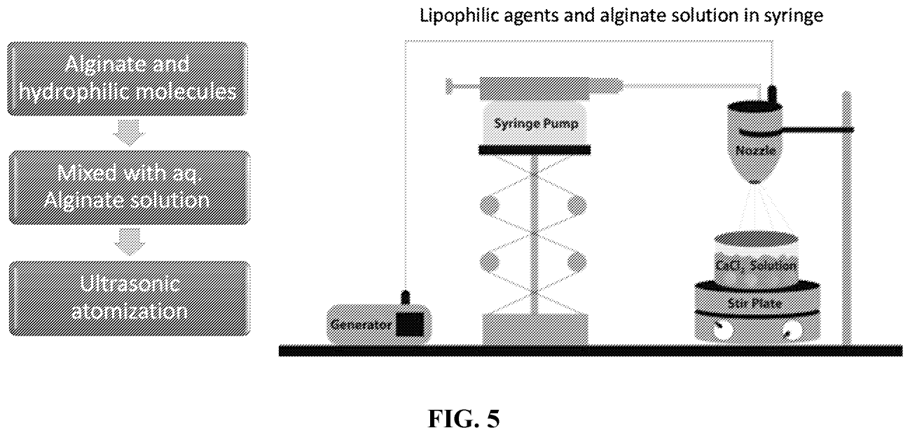

LEAMs have been shown to be very stable in different suspension solutions (e.g., water and saline) over several weeks. The LEAMs make an excellent radiographic contrast agent due to their very high lipiodol content, about 33 wt % lipiodol. This lipiodol content is very high because the lipiodol is an integral component of particular volumes of the microsphere. This is very different than a lipiodol simply being attached to the surface of the microsphere. The spheres are visualized during delivery to the tumor site. The embolized site can be visualized post-treatment by CT imaging to confirm local drug delivery as shown in FIG. 4.

The inspiration behind pursuing this concept was to address the fundamental deficits of both lipiodol and embolic microspheres and combining their respective advantages. Lipiodol presents the advantages of being radiopaque and as a carrier for hydrophobic drugs and may be emulsified in combination with aqueous therapeutic agents; however, free (nonencapsulated) lipiodol presents the possibility of complications such as migratory fat emboli and/or thyrotoxicity from dissociated iodine. For example, lipiodol when injected intra-arterial, is only partially retained at the distal end of the arteriole, and a significant amount of lipiodol escapes and moves to the subsequently encountered capillary bed such as the lungs. When lipiodol was radiolabeled with I-131 and injected intra-arterially into sarcoma tumors of the extremities, the main complication was that lipiodol escaped from the tumor and went to the lungs (Richardson et al., Can Med Assoc J. 1966, 94:1086-91). To limit lung activity, patients had to remain at bedrest without extremity movement for several days. The lipiodol within microspheres will be well-retained at the intended site of embolization while simultaneously allowing excellent visualization of the localization of the lipiodol microspheres when administered with therapeutic intent.

Therapeutic applications of these LEAMs include but are not limited to: (i) Visualization of embolic agents, used as therapy for bleeding gastrointestinal sites or other regions of bleeding to confirm site embolization (Vollherbst et al. PLoS One. 2018, 13: e0198911). (ii) Accurate radiographic visualization for drug-eluting spheres or radioembolic spheres delivered for treatment of tumors, such as liver tumors, with therapeutic radionuclides. This need for accurate radiographic visualization of microspheres was the initial motivation for the discovery of LEAMs as the inventors required better understanding of the location of the LEAMs while carrying out pre-clinical studies of liposomal alginate microspheres (LAMs). (iii) Construction of Mega-LEAMs (2-5 millimeters in size) containing multimodal imaging agents including but not limited to lipiodol and diagnostic radionuclides for accurate placement in radiographic or CT localization at the site of the tissue to be removed during surgery and intraoperative markers such as radionuclides for detection with a scintillation probe to guide the surgeon to the targeted tissue to be removed.

Mega-LEAMs. Colored guided surgery and radioprobe-guided surgery have recently become a very important area of clinical application and surgical research (van Leeuwen et al. J Nucl Med. 2020, 61:13-19). Radioprobe-guided surgery are currently performed with I-125 containing seeds. I-125 has significant disadvantages due to its long half-life and specialized manufacturing process requiring long lead times prior to use. I-125 also has a low gamma energy so that simultaneous use of ^{99m}Tc sulfur colloid to detect sentinel lymph nodes can cause downscatter radiation that interferes with detection of the I-125 seed localization (Hung T J, et al, Curr Radiopharm. 2017 Aug. 24; 10 (2): 111-114, Al-Hilli et al. Ann Surg Oncol (2015) 22:3350-3355). With LEAMs it is possible to use a wide variety of radionuclides with high energies that would not interfere with sentinel node localization.

As mentioned earlier, it is also possible to manufacture large-sized (2-5 mm) lipiodol microspheres containing multiple imaging agents (Mega-LEAMs) that can be used as radiographic markers of tumor nodules and masses as described in a recent review article by van Leeuven et al. (J Nucl Med. 2020, 61:13-19). In certain aspects these Mega-LEAMs can contain three different agents including (1) lipiodol, (2) a radionuclide, and (3) a fluorescent marker. The lipiodol is important for accurate pre-surgical localization by CT or mammography placement of the Mega-LEAMs. The intra-operative localization can be done by radionuclides and fluorescent markers also encapsulated in the Mega-LEAMs that guide the surgeon during the performance of the surgery. The radionuclides guide the surgeon to the region of the surgery using hand-held radioprobe localization once the visual fluorescent markers for intra-operative surgical guidance for removal of breast tumors specifically targeted lymph nodes, bowel lesions that are localized by endoscopy, etc.

Currently, free blue dye and radioactive nanoparticles are used to locate sentinel lymph nodes by a radio-guided probes and the surgeon's visual recognition. In addition, iodine-125 seeds, which emit low energy gamma photons are being used for radiographic placement into breast tumors which allow the surgeon to detect the precise location of the breast tumor by hand-held radioprobe detection during the surgery to ensure removal of the radiographically localized breast tumor. Disadvantages of the I-125 seed include its costliness, its radiation safety hazard due to its 50 day half-life, its lack of visual color or fluorescence to help the surgeon visually locate the lesion to be removed, and its low gamma photon energy 27 keV which scatter from ^{99m}Tc agents (140 keV) used to localize the sentinel lymph node.

Other uses of Mega-LEAMs for surgical guidance include a method to radiographically and color mark suspicious lesions encountered during gastrointestinal endoscopy. The goal would be to mark these suspicious lesions by injection into the site of the lesion during the endoscopic procedure and then during surgery, the surgeon would be guided to the site of the lesion for surgical removal of the intestine in this region. Mega-LEAMs offer the potential to solve these surgical problems by allowing for detection of the general region with a radioguided hand-held probe and then visual or fluorescent confirmation during the surgery.

The microsphere contains an oil-in-water emulsion using lipiodol as the oil phase and alginate solution as the aqueous phase. Before the combination of the two phases, it is best to add additional dyes or compounds which can stabilize the emulsion (Sudan red for the lipiodol phase, or doxorubicin for the aqueous phase). The emulsion of the two phases may be prepared by shearing forces either from magnetic stirring or via dual-syringe exchanging. Droplet size is directly correlated with shearing force. Minute amounts of surfactant may be incorporated into the emulsion to stabilize and decrease oil droplet size. An example of a ratio of lipiodol to alginate solution is 1:1.25. 1:1.5, 1:1.75, 1:2, 1:2.25, 1:2.5, 1:2.75, or any ratio there between. In certain aspects the concentration of alginate solution is 1, 2, 3, 4, to 5% w/v.

The absence of a surfactant (particularly Tween 80) does not allow for consistent stabilization of the emulsion necessary for sphere formulation. For standardize production and broaden the clinical applications of LEAMs, a surfactant (e.g., Tween 80) is needed and which will allows for the incorporation of other anti-tumor/chemotherapeutic agents. In certain aspects, prior to emulsification, the oil phase includes the oil phase (e.g., lipiodol), the hydrophobic agent of choice (dye, radiochelation complex, drug), and a surfactant, (e.g., Tween80).

Once emulsified, the solution is fashioned into microspheres. In one example, the method utilizes an ultrasonicator nozzle (e.g., Sono-tek ultrasonicator) for the atomization of the emulsion solution into droplets of relatively homogenous size. The solution is fed into the activated nozzle via syringe pump. The nozzle, which is suspended over a solution of CaCl₂, atomizes the solution into droplets which can then fall into the CaCl₂) solution. The alginate in the droplets immediately crosslink with the Ca2+ ions, causing the gel droplets to solidify and retain the emulsion. (nozzle settings can be, for example, a frequency of 25 KHz, a power of 5.0 W, a flow rate of 0.5 ml/min, and a nozzle height of 3 cm).

Prior to administration to a subject, spheres may be filtered via sieving to achieve a preferred sphere diameter. The spheres can be introduced to a target, e.g., a liver tumor, intrarterially via a catheter. Given that the diameter of capillaries is ˜10 μm, the size of the spheres should be greater to achieve embolization; also the spheres must be small enough to penetrate past the arteriolar level to the reach the capillary level. In certain examples, the average sphere size generated is ˜20, 30, 40, to 50 μm, including all values and ranges there between.

Proof of concept has been demonstrated for LEAMs as a carrier for hydrophobic radiochelation complexes for radiodiagnostic or radiotherapeutic purposes. LEAMs have been generated that carry the hydrophobic chelation complex indium-111 oxine. To demonstrate a proof of concept, 500 μL of saline solution containing 150 μCi of indium-111 oxine were emulsified with 1 ml of lipiodol containing trace amounts of Tween80. The solution was further emulsified with an additional 1.5 ml of 1.5% alginate solution. Upon successful emulsion of the two phases, the microspheres were generated via drop-wise descension into a solution of CaCl₂). The resulting microspheres underwent several washes and incubation for over 24 hrs. Final radiolabeling efficiency proved to be >94% (accounting for decay). The inventors conclude from this experiment that the hydrophobic chelation complex was extracted into the oil phase of the LEAMs. The oil phase being guarded in an emulsified state inside the LEAMs is entrapped in the LEAMs. Although, this phenomenon was demonstrated with the generation of indium-111 oxine loaded LEAMs, this hydrophobic-radionuclide-chelation complex encapsulated in LEAMs can be applied to other hydrophobic radionuclide chelation complexes including but not limited to Re-188-HDD or Cu-64-DOTA-triarginine-lipid constructs (Lamber et al. Eur J Nucl Med Mol Imaging 33 (3): 344-52, 2006; Wang et al. Nanotheranostics 4 (3): 142-55, 2020), and Ga-68 oxine, Ga-68 tropolone, and Ga-68 mercapropyridine-oxide (MPO) (Yano et al. J Nucl Med 26:1429-37, 1985).

Alginate microspheres are produced via ultrasonication and can contain oil-in-water emulsions. Using lipiodol as the oil phase allows for the alginate spheres to be radiopaque with a reduced risk of sequelae secondary to migratory fat emboli. Ultrasonication as a means of microsphere production is a recent concept and particularly helpful in the production of a sphere which carries both hydrophilic and hydrophobic phases. Traditional methods such as microfluidics and water-in-oil emulsification are not expected to be applicable manufacturing methods for the production of LEAMs, due to the potential disruption of the emulsion via the presence of an aqueous and hydrophobic phase as crucial components of production. Indeed, it is difficult to generate alginate microspheres which carry both hydrophilic and hydrophobic contents using the traditional methods. Ultrasonication is arguably a superior method for the production of LEAMs as it can achieve proper size and maintain the emulsion dispersion. Additionally, methods such as microfluidic devices, which are praised for their precision in sphere diameter, may take hours to produce an adequate amount and are prone to malfunction and occlusion. Ultrasonication for the production of microspheres takes less than an hour.

Advantages of LEAMs include: (i) Degradability. LEAMs are made of alginate, a natural polymer that is considered safe for human use and has already been used as a drug delivery agent. LEAMs incorporate both degradability and radiopacity. (ii) Multimodal imaging. LEAMs containing lipiodol are effective contrast agents for CT imaging. An understanding of the temporal and spatial distribution of embolic microspheres is clinically beneficial. Multimodal imaging allows for efficiency and standardization of embolization endpoints. LEAMs can effectively carry lipiodol and other lipophilic dyes as well as one example of an essential oil. (iii) Highly effective lipophilic agent carrying capacity. The lipid emulsion of the LEAM can include a 1:2 ratio of oil to water throughout the alginate microsphere. This results in a very high amount of lipophilic agent carrying capacity. This differs from the more limited carrying capacity of other describe drug eluting microspheres. This method of drug carrying by the LEAMs appears to have not been previously described in the research literature for production of embolic microspheres. The development of endovascular embolization includes two categories of drug loading into microspheres: (1) adsorption of drugs by ionic interactions and (2) sponge-like microspheres that “soak up” the drug. An important drawback of these methods was that the size, rigidity and elasticity vary as a function of their drug loading leading to unpredictability in the level of the vascular tree where embolization occurs. The currently described LEAMs use a completely different loading process with the formation of a lipid emulsion. (iv) Ease of manufacture of a wide variety of size ranges. The small microspheres can be manufactured with a ultrasonication nozzle and are within the size range anticipated for embolic therapy (30 microns-700 microns). The larger sized Mega-LEAMs (which are produced in the mm-size range) have potential for surgical localization by using the CT contrast of the lipiodol for accurate placement of the LEAM under radiographic guidance into tissue planned for surgical removal, and then using a hand held nuclear probe for general surgical localization and a fluorescent optical agent for more specific surgical localization. Due to the different size-dependent applications which may employ LEAMs, the ability to easily produce spheres of variable size is beneficial.

The LEAMs can be used to carry lipiodol for radiographic distribution monitoring of simple embolic microspheres, monitoring of the distribution of radiotherapeutic microspheres, multimodal imaging enabling CT contrast, radioprobe and fluorescent imaging for both embolic therapy and for pre-surgical tumor localization.

1. An alginate microsphere comprising a crosslinked alginate microsphere containing water in oil emulsion portions encapsulated by the crosslinked alginate.

2. The alginate microspheres of claim 1, wherein the emulsion comprises a surfactant.

3. The alginate microspheres of claim 2, wherein the surfactant is polyoxyethylene sorbitan monooleate (TWEEN 80®), oleyl polyoxyiglycerides, polyoxyethylated castor oil, caprylic capric triglycerides, glyceryl monocaprylocaprate, and mixtures thereof.

4. The alginate microsphere of any one of claims 2 to 3, wherein the surfactant is present at an amount of 20 to 65 weight percent.

5. The alginate microsphere of any one of claims 1 to 4, wherein the oil is a fatty acid.

6. The alginate microsphere of claim 5, wherein the fatty acid is an ethiodized fatty acid.

7. The alginate microsphere of claim 6, wherein the ethiodized fatty acid is lipiodol.

8. The alginate microsphere of any one of claims 1 to 7, wherein the emulsion is 20% to 60% oil.

9. The alginate microsphere of claim 6, wherein the iodine moiety comprises iodine 131 and/or iodine 125.

10. The alginate microspheres of any one of claims 1 to 9, wherein the microspheres have an average diameter of 20 microns to 2000 microns.

11. The alginate microspheres of any one of claims 1 to 9, wherein the microspheres have an average diameter of 2 to 5 millimeters.

12. The alginate microspheres of any one of claims 1 to 9, wherein the microspheres have an average diameter of 50+/−10 microns.

13. The alginate microsphere of any one of claims 1 to 12, further comprising a hydrophobic solute in the emulsion portion.

14. The alginate microsphere of claim 13, wherein the hydrophobic solute is a hydrophobic therapeutic agent or dye.

15. The alginate microsphere of claim 14, wherein the hydrophobic dye is a fluorescent dye.

16. The alginate microsphere of claim 14, wherein the therapeutic agent is a radionuclide or chemotherapeutic.

17. The alginate microsphere of claim 16, wherein the radionuclide is a hydrophobic radionuclide chelate.

18. The alginate microsphere of any one of claims 1 to 17, further comprising a hydrophilic solute in the emulsion portion.

19. The alginate microsphere of claim 18, wherein the hydrophilic solute is a hydrophilic drug or dye.

20. The alginate microsphere of claim 19, wherein the hydrophilic dye is a fluorescent dye.

21. The alginate microsphere of claim 19, wherein the hydrophilic drug is a radionuclide or chemotherapeutic.

22. The alginate microsphere of claim 21, wherein the radionuclide is a hydrophilic radionuclide chelate.

23. An alginate microsphere comprising a crosslinked alginate microsphere containing a radiopaque emulsion encapsulated by the crosslinked alginate.

24. The alginate microsphere of claim 23, wherein the emulsion comprises a fatty acid.

25. The alginate microsphere of claim 23, wherein the fatty acid is an ethiodized fatty acid.

26. The alginate microsphere of claim 25, wherein the ethiodized fatty acid is lipiodol.

27. The alginate microsphere of claim 25, wherein the iodine moiety comprises iodine 131.

28. A method for producing an alginate microsphere encapsulating a radiopaque emulsion comprising:
(i) preparing an ethiodized fatty acid emulsion;
(ii) homogenizing the emulsion in an alginate solution; and
(iii) forming the alginate microsphere encapsulating a radiopaque emulsion microsphere by contacting emulsion droplets with a calcium chloride solution.

29. The method of claim 28, wherein the ethiodized fatty acid is lipiodol.

30. The method of claim 28, wherein the step of forming the alginate microsphere include ultrasonic atomization of the emulsion in alginate solution.
